(19) United States
(12) Patent Application Publication
(10) Pub. No.: US 2017/0233276 A1
(43) Pub. Date: Aug. 17, 2017

(54) METHOD FOR THE MANAGEMENT OF BIOLOGY IN A BATCH PROCESS

(71) Applicant: AnaeCo Limited, Bentley, Western Australia (AU)

(72) Inventors: Lee Richard WALKER, High Wycombe, Western Australia (AU); Ralf CORD-RUWISCH, Murdoch, Western Australia (AU); Wipa CHARLES, Shelly, Western Australia (AU)

(73) Assignee: AnaeCo Limited, Bentley, Western Australia (AU)

(21) Appl. No.: 15/521,696

(22) PCT Filed: Oct. 28, 2015
(86) PCT No.: PCT/AU2015/000647

§ 371 Date: Apr. 25, 2017

(30) Foreign Application Priority Data
Oct. 28, 2014 (AU) 2014904316

(51) Int. Cl. C02F 11/04; C02F 11/12; C02F 3/28; C02F 3/30; C12P 5/02; C05F 17/00

(52) U.S. Cl. CPC C02F 11/04; C12P 5/023; C05F 17/0036; C02F 3/28; C02F 3/301; C02F 11/12; C02F 2209/16; C02F 2301/046

(57) ABSTRACT

A method for the treatment of organic waste, the method comprising alternating steps of anaerobic digestion and aerobic composting conducted in a single reactor vessel, wherein at or about the completion of the anaerobic digestion step at least a portion of any free draining fluid from the reactor vessel is directed for reuse in subsequent anaerobic digestion steps, and solids remaining in the reactor vessel from the anaerobic digestion step are subjected to a dewatering step from which a liquid is obtained that is ultimately also directed, at least in part, for reuse in subsequent anaerobic digestion steps. A method for the management of biology in a batch process, wherein the batch process is an anaerobic digestion process, is also described.

### FIELD OF THE INVENTION

The present invention relates to a method for the management of biology in a batch process. More particularly, the method of the present invention is intended for use in anaerobic digestion of organic waste. This organic waste is in one form the organic component of a municipal solid waste.

The present invention further relates to a process or method for the treatment of organic waste, the process comprising alternating steps of anaerobic digestion and aerobic composting conducted in a single reactor vessel.

More particularly, the present invention also describes populations of methanogenic microorganisms that are present in specific phases of material present in and produced during the anaerobic step of the process for the treatment of organic waste. Also described is the treatment of those populations in the management of the process or method of the present invention.

### BACKGROUND ART

The treatment of mixed municipal solid waste (“MSW”) presently most typically comprises passing that waste to some form of separation process by which organic materials therein are first separated, as much as possible, from inorganic materials. This initial separation step is invariably a size based separation, with organic material typically being smaller or softer than much of the inorganic material. The organic materials are subsequently directed, at least in part, to a biological stabilisation or degradation process, whilst the inorganic material is sorted into recyclables and non-recyclables, the latter being passed to landfill. The product of the biological stabilisation or degradation process is ideally a compost material and/or a biogas.

Typically, systems for the biodegradation of organic waste material are directed to either aerobic or anaerobic processes. However, there are a small number of systems that have sought to combine both anaerobic and aerobic biodegradation processes. The processes of German Patent 4440750 and International Patent Application PCT/DE1994/000440 (WO 1994/024071) each describe the combination of an anaerobic fermentation unit and an aerobic composting unit. Importantly, these systems describe discrete and separate vessels for the aerobic and anaerobic biodegradation processes.

It is known that solid organic waste material may be treated under either anaerobic or aerobic conditions to produce a bioactive, stable end product that, for example, may be used as compost for gardens. This process is achieved through the action of, respectively, anaerobic or aerobic microorganisms that are able to metabolise the organic waste material to produce the bioactive, stable end product.

It is also known that the aerobic decomposition of solid organic waste material takes place in the presence of oxygen. The temperature of the waste material rises as some of the energy produced during aerobic decomposition is released as heat, often reaching temperatures of approximately 75° C. under ambient conditions. The solid end product is often rich in nitrates which are a readily bio-available source of nitrogen for plants, making the end product particularly suitable as a fertiliser.

It is further known that the anaerobic digestion of solid organic waste material takes place in the absence of oxygen. Anaerobic microbial metabolism is understood to be optimised when the organic material is heated to temperatures at which mesophilic or thermophilic bacteria are operative. The process of anaerobic microbial metabolism results in the production of biogas, in turn predominantly methane and carbon dioxide. The solid product of the process is often rich in ammonium salts. Such ammonium salts are not readily bio-available and are, consequently, generally treated under conditions in which aerobic decomposition will occur. In this manner the material is used to produce a product that is bio-available.

International Patent Application PCT/AU00/00865 (WO 01/05729) describes an improved process and apparatus in which aerobic and anaerobic processes are combined for the treatment of the organic fraction of MSW (OFMSW), and in which many of the inefficiencies of the previous processes and apparatus are overcome. The process and apparatus are characterised at a fundamental level by the sequential treatment of organic waste material in a single vessel, through an initial aerobic step to raise the temperature of the organic waste material, an anaerobic digestion step and a subsequent aerobic treatment step. During the anaerobic digestion step a process water or inoculum containing microorganisms is introduced to the vessel to create conditions suitable for efficient anaerobic digestion of the contents and the production of biogas. The introduced inoculum also aids in heat and mass transfer as well as providing buffer capacity to protect against acidification. Subsequently, air is introduced to the residues in the vessel to create conditions for aerobic degradation. It is further described that the water introduced during anaerobic digestion may be sourced from an interconnected vessel that has undergone anaerobic digestion.

The sequential treatment of organic waste material in a single vessel requires that the process be conducted as a batch process. Whilst the single vessel process described in International Patent Application PCT/AU00/00865 (WO 01/05729) provides many advantages with respect to prior art processes, it does create challenges in maintaining process stability during anaerobic treatment. Amongst these is an inability to control the rate of organic acid generation during early anaerobic digestion.

The microorganisms employed during anaerobic digestion of the biomass typically comprise a delicate balance of “acid producing” and “acid consuming” micro-organisms. For example, in an uninoculated system the number of acid producing micro-organisms typically exceed the number of acid consuming micro-organisms.

Acid producing bacterial species will produce organic acids which will typically cause the pH of a decomposing biomass to drop (become more acidic). Acid consuming microbial species contribute to the production of biogas, including methane, and cause the pH to rise (become more alkaline or basic). Early in a typically batch anaerobic digestion, the number of organic acid producing bacteria exceed those that consume these acids. This imbalance can result in acidification, process instability and/or process failure and highlights the need for accurate monitoring of the process.

Similarly, the introduction of microorganisms to the reactor is not something that can readily be monitored when the process is being conducted on a commercial scale and in real time. In the process of International Patent Application PCT/AU00/00865 (WO 01/05729) the liquid produced during the anaerobic phase of decomposition is re-used. As such, the process is re-exposed to what has been produced in that earlier anaerobic phase and is present in the liquid that has been re-used. Consequently, the conditions in the reactor may become too acidic over time. This is particularly the case if the level of volatile fatty acids (VFAs) is rising due to incomplete microbial exhaustion of the VFA present in the liquid from a previous batch prior to reintroduction to the reactor. The postulated decrease in pH may eventually lead to process failure.

Similarly, temperature management of static high solids batch anaerobic digestion processes becomes difficult due to poor mixing and inefficient mass transfer. The ensuing unfavourable conditions may also provide poor microbial performance, such as a decrease in the metabolism of the microorganisms as a result of lower temperature. In turn, the performance of the degradation process and the production of biogas are hampered.

The process and apparatus of Application PCT/AU00/00865 (WO 01/05729), in which aerobic and anaerobic processes are combined for the treatment of OFMSW, are further described in several further International Patent Applications, including Applications PCT/AU2012/000738 (WO 2013/003883), PCT/2012/001057 (WO 2013/033772) and PCT/AU2012/001058 (WO 2013/033773), for example. These POT applications describe different aspects of the process and/or apparatus first described, in a relatively fundamental and formative form, in Application PCT/AU00/00865 (WO 01/05729).

Wagner et al. have published a study in which they have examined anaerobic digestion of biological waste, biogas production and the impact of fatty acid levels thereon (Wagner et al., Effects of various fatty acid amendments on a microbial digester community in batch culture, Waste Management 31 (2011) 431-437). The aim of this study seems to have been a desire to understand the influence of substrate composition on the microorganisms involved in anaerobic digestion. It was observed that the particular anaerobic digester or biogas reactor from which the samples were taken for this study contained at least the species Methanoculleus sp, and Methanothermobacter wolfei (M. wolfei). Both species were identified as having an important role in digester performance. The authors further observed that only a small number of species played a significant role in biogas production. This study, and others that have preceded it, have as their focus the study of specific established anaerobic microbiological populations and how they influence biogas production and/or how their biogas production is influenced by substrate fluctuations and forms.

As noted hereinabove, the prior art has largely been directed to either aerobic or anaerobic processes; not methods in which both aerobic and anaerobic processes take place in the one reactor. With both processes taking place in the one reactor it raises the challenge of how to maintain appropriate biology for the efficient operation of at least the anaerobic digestion process.

The chemistry of the anaerobic digestion of organic material and the production of biogas is in many respects well understood. However, as noted hereinabove, the specific microorganisms are not well known, nor is it well understood how they contribute to the process of anaerobic digestion. It is thought that two main methanogenic microorganisms are typically present in anaerobic digestion processes, being hydrogen consumers and acetate consumers, and that efficient operation of an anaerobic digester requires both to be present. The acetate consuming microorganisms are generally thought to be delicate and more sensitive to variations in environmental conditions, whilst the hydrogen consumers are more robust, in particular, being more resistant to increased levels of ammonia. The biology of the process described in International Patent Application PCT/AU00/00865 (WO 01/05729) is understood to operate with relatively high levels of ammonia present as this is used to buffer the process, which is in turn necessary due to the batch nature of the process (referring to the sudden and rapid production of VFA that occurs soon after the commencement of the anaerobic digestion phase). This high ammonia level results in acetate consumers struggling and the hydrogen consumers being more successful. This is counterintuitive as traditionally it is understood that methanogenic microbiological populations are split along the lines of roughly 70% being acetate consumers and 30% hydrogen consumers.

The method for the treatment of organic waste, and the anaerobic digestion, of the present invention have as one object to overcome substantially the abovementioned problems of the prior art, or to provide a useful alternative thereto.

The preceding discussion of the background art is intended to facilitate an understanding of the present invention only. The discussion is not an acknowledgement or admission that any of the material referred to is or was part of the common general knowledge as at the priority date of the application.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

Throughout the specification and claims, unless the context requires otherwise, the term “body of organic material”, variations thereof, or the term Organic Fraction of Municipal Solid Waste (OFMSW), will be understood to imply an organic mass, body or component, composed of man-made or natural organic material. Such may include food, kitchen, animal, garden, vegetable or other putrescible material suitable for anaerobic and aerobic action, the by-products of which are at least a gas, more specifically a biogas, and a composted, carbon reduced end product, water and inoculum. The biogas may comprise at least hydrocarbons such as methane and ethane, carbon dioxide, hydrogen, nitrogen, oxygen, and sulphurous gases such as hydrogen sulphide in any ratio.

### DISCLOSURE OF THE INVENTION

In accordance with the present invention there is provided a process or method for the treatment of organic waste, the process comprising alternating steps of anaerobic digestion and aerobic composting conducted in a single reactor vessel, wherein at or about the completion of the anaerobic digestion step at least a portion of any free draining fluid from the reactor vessel is directed for reuse in subsequent anaerobic digestion steps, and solids from the anaerobic digestion step remaining in the reactor vessel are subjected to a dewatering step from which a liquid is obtained that is ultimately also directed, at least in part, for reuse in subsequent anaerobic digestion steps.

Preferably, both the free draining fluid from the reactor vessel and the liquid obtained from the dewatering step contain methanogenic microorganisms that contribute to the anaerobic digestion of organic waste.

Still preferably, the free draining fluid contains hydrogen consuming microorganisms. The liquid obtained from the dewatering step contains acetate consuming microorganisms.

In one form of the present invention the methanogenic microorganisms contained in the free draining liquid includes at least one Methanoculleus species. Preferably, the at least one Methanoculleus species includes at least one of Methanoculleus thermophilus, Methanoculleus chikugoensis and Methanoculleus submarinus.

Still preferably, the free draining liquid further includes at least one Methanothermobacter or Methanobacterium species, such as Methanothermobacter wolfeli.

In one form of the present invention the methanogenic microorganisms contained in the liquid obtained from the dewatering step includes at least Methanosarcina thermophila.

Preferably, the methanogenic microorganisms contained in the liquid obtained from the dewatering step further includes Methanoculleus thermophilus.

The Total Ammonium Nitrogen concentration during anaerobic digestion is preferably maintained at less than about 3,000 mg/L, for example at about 2,000 mg/L).

In accordance with the present invention there is further provided a method for the management of biology in a batch process, wherein the batch process is an anaerobic digestion process and at or about the completion of a first anaerobic digestion step at least a portion of any free draining fluid from the reactor vessel in which the anaerobic digestion step is conducted is directed for reuse in subsequent anaerobic digestion steps, and solids from the anaerobic digestion step remaining in the reactor vessel are subjected to a dewatering step from which a liquid is obtained that is ultimately also directed, at least in part, for reuse in subsequent anaerobic digestion steps.

Preferably, both the free draining fluid from the reactor vessel and the liquid obtained from the dewatering step contain methanogenic microorganisms that contribute to the anaerobic digestion of organic waste.

Still preferably, the free draining fluid contains hydrogen consuming microorganisms. The liquid obtained from the dewatering step contains acetate consuming microorganisms.

Still further preferably, the free draining fluid from the reactor vessel and the liquid obtained from the dewatering step are stored separately, thereby allowing the preparation of a specific inoculum blend that can be adjusted to meet the needs of a specific feedstock. In this manner the balance of hydrogen consuming and acetate consuming microorganisms may be prepared specifically depending upon the composition of a particular feedstock.

Preferably, the Total Ammonium Nitrogen concentration during anaerobic digestion is maintained at less than about 3,000 mg/L, for example at about 2,000 mg/L). In one form of the present invention the methanogenic microorganisms contained in the free draining liquid includes at least one Methanoculleus species. Preferably, the at least one Methanoculleus species includes at least one of Methanoculleus thermophilus, Methanoculleus chikugoensis and Methanoculleus submarinus.

Still preferably, the free draining liquid further includes at least one Methanothermobacter or Methanobacterium species, such as Methanothermobacter wolfeii.

In one form of the present invention the methanogenic microorganisms contained in the liquid obtained from the dewatering step includes at least Methanosarcina thermophila.

Preferably, the methanogenic microorganisms contained in the liquid obtained from the dewatering step further includes Methanoculleus thermophilus.

In one form of the present invention a portion of the dewatered solids remaining in the reactor vessel from anaerobic digestion is directed for reuse in subsequent anaerobic digestion steps.

Preferably, between about 5 to 20% by weight of the dewatered solids remaining in the reactor vessel from anaerobic digestion is directed for reuse. Still preferably, about 10% by weight of the dewatered solids remaining in the reactor vessel from anaerobic digestion is directed for reuse.

### BEST MODE(S) FOR CARRYING OUT THE INVENTION

The present invention provides a process or method for the treatment of organic waste, the method comprising alternating steps of anaerobic digestion and aerobic composting conducted in a single reactor vessel, wherein at or about the completion of the anaerobic digestion step at least a portion of any free draining fluid from the reactor vessel is directed for reuse in subsequent anaerobic digestion steps, and solids from the anaerobic digestion step remaining in the reactor vessel are subjected to a dewatering step from which a liquid is obtained that is ultimately also directed, at least in part, for reuse in subsequent anaerobic digestion steps.

Both the free draining fluid from the reactor vessel and the liquid obtained from the dewatering step contain methanogenic microorganisms that contribute to the anaerobic digestion of organic waste. The free draining fluid largely contains hydrogen consuming methanogenic microorganisms whilst the liquid obtained from the dewatering step largely contains acetate consuming methanogenic microorganisms.

The methanogenic microorganisms contained in the free draining liquid include at least one Methanoculleus species. For example, the at least one Methanoculleus species comprises one or more of Methanoculleus thermophilus, Methanoculleus chikugoensis and Methanoculleus submarines.

The free draining liquid further includes at least one Methanothermobacter or Methanobacterium species, such as Methanothermobacter wolfeii.

The methanogenic microorganisms contained in the liquid obtained from the dewatering step includes at least Methanosarcina thermophila. The methanogenic microorganisms contained in the liquid obtained from the dewatering step further includes Methanoculleus thermophilus.

The present invention further provides a method for the management of biology in a batch process, the batch process being an anaerobic digestion process that is effectively a part or portion of the process for the treatment of organic waste material as described herein.

In International Patent Application PCT/AU00/00865 (WO 01/05729), the entire content of which is incorporated herein explicitly by reference, there is described a process and apparatus in which aerobic and anaerobic processes are combined for the treatment of the organic fraction of MSW (OFMSW) and within the context of which the present invention may operate and provide particular advantages.

The process and apparatus of International Patent Application PCT/AU00/00865 (WO 01/05729) are characterised at a fundamental level by the sequential treatment of organic waste material in a single vessel, through an initial aerobic step to raise the temperature of the organic waste material, an anaerobic digestion step and a subsequent aerobic treatment step.

During the anaerobic digestion step a process water or inoculum containing micro organisms is introduced to the vessel to create conditions suitable for efficient anaerobic digestion of the contents and the production of biogas. The introduced inoculum also aids in heat and mass transfer as well as providing buffer capacity to protect against acidification. Subsequently, air is introduced to the residues in the vessel to create conditions for aerobic degradation. It is further described that the water introduced during anaerobic digestion may be sourced from an interconnected vessel that has undergone anaerobic digestion.

The sequential decomposition process of organic waste material is a two stage process including an anaerobic digestion stage followed by an aerobic composting stage. Preferably, the organic waste material undergoes a preliminary aerobic composting pre-conditioning stage followed by a preliminary digestion pre-conditioning stage before commencement of the anaerobic digestion stage and the aerobic composting stage.

Biogas is produced at the commencement of and during the anaerobic digestion stage. A mixture of methane and oxygen in the vessel would provide a combustible and potentially explosive gas mixture. Furthermore, the introduction of an anaerobic inoculum into a vessel having a moderate to high oxygen level is undesirable for the anaerobic inoculum since many anaerobic microorganisms are intolerant to oxygen.

Thus, it is an advantage of the preliminary anaerobic digestion pre-conditioning stage to deplete oxygen levels in the sealed vessel before commencement of the anaerobic digestion stage.

When the oxygen level drops to below accepted standards (for example less than 1% oxygen) the anaerobic digestion stage of the sequential decomposition process can commence.

The anaerobic digestion stage comprises the steps of: 1) adjusting the moisture content of the waste material to about 50 to 95% wet weight (w/w); and 2) digestion of the waste material by anaerobic and facultative microorganisms.

Water from an external source at the second port is received through the second recirculation line and pumped by the second pump into the vessel via the control line and the feeder lines. The feeder lines evenly distribute the water through the organic waste material such that the moisture content of the waste material ranges from 50 to 95% wet weight (w/w) throughout the contents of the vessel. It will be appreciated that the water from the external source is preferably water removed from another vessel which has undergone the anaerobic digestion stage and is being recirculated by the second recirculation line into the present vessel. In this way, process water from one anaerobic digestion is used to inoculate the contents of an interconnected vessel undergoing the anaerobic digestion stage in a multiple vessel system.

The inoculation of the contents of an interconnected vessel with the process water from another vessel that has undergone anaerobic digestion is considered advantageous by the Applicants due to the prior adaptation of the microorganisms contained therein to the relevant substrate, being the organic waste food source, and process conditions, including temperature, salinity, degree of osmotic stress and total ammonium nitrogen (TAN) concentration.

The anaerobic digestion stage operates in a mesophilic to thermophilic temperature range between about 15° C. to 75° C., preferably over 50° C., for a period between about 4 to 20 days. Methane and carbon dioxide gases are generated during the anaerobic digestion stage. They are extracted under pressure through the gas extraction line and delivered to the de-watering tank where water is removed from the extracted gases. The extracted gases are then delivered through the first recirculation line to the gas storage tank via the first storage line. The gas may then be converted to electrical power by the power generator, or alternatively, used to heat water in the water heater tank.

The water which is removed from the extracted gases in the de-watering tank is then delivered to the heater tank by the de-watering line. The water may be heated in the water heater tank. The heated water may also be recirculated by the second recirculation line, the control line and the feeder lines back into the vessel for a subsequent anaerobic digestion stage, of another batch of organic waste material. In this way the heat and electricity indirectly generated by the anaerobic digestion stage can be utilised to subsidise energy requirements in interconnected vessels or used in subsequent stages of the sequential decomposition process occurring at a later time in the same vessel. It has been found that during the anaerobic digestion stage the amount of volatile solids is reduced and nitrogen content in the contents of the vessel is concentrated.

Following completion of the anaerobic digestion stage conditions within the vessel are altered such that the aerobic composting stage may commence.

The Applicants have undertaken studies that have revealed that the anaerobic microorganisms responsible for digestion of the organic waste in the anaerobic digestion step comprise both hydrogen consuming and acetate consuming methanogenic species, and importantly that the hydrogen consuming methanogenic species are largely present in free draining water obtained from the anaerobic digestion step and the acetate consuming methanogenic species are largely present in a slurry from the anaerobic digestion step.

The hydrogen consuming methanogenic species have been found to grow rapidly, whilst the acetate consuming methanogenic species grow slowly. That the Applicants have identified this and developed methods by which; in particular the acetate consuming methanogenic species can be harvested; has allowed the process of the present invention to be operable in an efficient manner.

The Applicants have identified that the hydrogen consuming microorganisms are more resistant to increased levels of total ammonium nitrogen (TAN) in the anaerobic digestion step than are the acetate consuming microorganisms. These increased levels of ammonium ions allow for a substantial carbonic acid—hydrogen carbonate ion buffer system to develop and maintain stable pH in the treatment system. As such, maintaining the population of acetate consuming microorganisms becomes particularly important in providing a commercially viable process for the treatment of organic waste.

The method of harvesting of the acetate consuming microorganisms for reuse in subsequent anaerobic digestion steps includes the dewatering of the solid, or sludge, product of anaerobic digestion. It is this combination that allows what are relatively short digestion steps and provides an overall treatment system with good longevity. This is understood to be more efficient than maintaining and introducing new microorganisms for each ‘batch’ anaerobic treatment step.

Dewatering of the solid, or sludge, product of anaerobic digestion described above may, in one form, be provided by an apparatus such as that described in International Patent Application PCT/AU2012/001055 (WO 2013/033770), the entire content of which is hereby incorporated by reference.

The Applicants have additionally determined that, upon completion of the anaerobic digestion period, a significant number of the methanogens are contained within the solids. These, as described above, are harvested from the material by dewatering of the solid or sludge product of anaerobic digestion. The resulting liquid contains both hydrogen consuming and acetate consuming methanogens. Importantly however, it is the main source of acetoclastic methanogens.

The dewatered solids are not devoid of methanogens, and significant quantities of methanogens remain within the dewatered solids. These methanogens are destined to be unloaded with the compost product of digestion and will be lost from the system. The Applicants propose that effective inoculation of a subsequent batch anaerobic digestion can be accomplished by transferring a quantity of these digested and dewatered solids (for example between about 5 to 20% by weight) into the ‘fresh’ material at the commencement of the anaerobic digestion period.

As the two key methanogens, one hydrogen consuming and the other acetate consuming, are predominantly contained within two distinct media, one free draining liquid and the other a silty slurry, pressed from the solids during dewatering, these inoculum sources may be kept separately. This will allow a management strategy based on the needs of the individual micro-organism. It would also then be possible to provide a specific inoculum blend that could be adjusted to meet the needs of a specific feedstock. That is, the balance of hydrogen consuming and acetate consuming microorganisms may be prepared specifically depending upon the composition of a particular feedstock.

The quantity of methane produced from an anaerobic digester, and the rate at which the material being digested can be stabilised, is related to the number of methane producing microorganisms present within the reactor. Typically, the stability and performance of an anaerobic digester can be enhanced by an increase in the number of methane producing microorganisms present, providing an increase in biogas production rate and a decrease in the time required to provide solids stabilisation. This is particularly true when considering the number of acetate consuming microorganisms that are present. Acetate consuming methane producing microorganisms (methanogens) are generally thought to be delicate and more sensitive to variations in environmental conditions and grow slowly. Furthermore, acetate consuming methanogens are closely associated with the solids being digested. Consequently, the number of methane producing microorganisms present in an anaerobic digester, and in particular the number of acetate consuming methanogens, can be increased by retaining, within a reactor, a quantity of the digested solids which is added to the incoming feed of a subsequent batch. Moreover, the number of methanogens present in an anaerobic digester can be increased by transferring a quantity of digested solids into a reactor containing the fresh feedstock of a subsequent batch just prior to the commencement of an anaerobic digestion process. The increase in the number of methanogenic microorganisms present allows the anaerobic digester to be smaller, have shorter hydraulic and solids retention times and maintain a stable population of methanogenic microorganisms when compared to systems without solids inoculation.

The transfer of solid inoculum (solid residues remaining at the end of an anaerobic digestion step) into a reactor being loaded with fresh incoming material has been found by the Applicants to be advantageous as the methane producing organisms present within the material have been found to survive the aerobic conditions that are present during the initial aeration period of the process and apparatus of International Patent Application PCT/AU00/00865 (WO 01/05729).

The quantity of digested solids transferred to, or retained within, a reactor as an inoculum, may in some embodiments of the present invention be a specific percentage, for example 5 to 20% or more by weight. In a preferred embodiment this percentage is about 10% by weight. In some embodiments this percentage may be, for example, 25% or more, or 50% or more by weight. However, the Applicants anticipate that the preferred range of solids to be used as an inoculum is between about 5 and 20% by weight.

Preliminary testing by the Applicants has identified that the transfer of residual solids remaining in the reactor at the completion of an anaerobic digestion period (20% by weight) into fresh incoming material (80% by weight) resulted in a 70% decrease in acetate accumulation (3,360 compared to 1,020 mg/L) during the initial anaerobic digestion period and an overall 17% decrease in the time required stabilise the material (9 compared to 7.5 days).

The present invention will now be described with reference to the following non-limiting example, in which the determination of the microbial population in the anaerobic phase of the above described process is set out.

### Example 1

The efficiency of the methanogenic microorganisms employed in the process of the present invention is measured via the methane generation rate of the methanogenic culture. The Applicants anticipate that this methane generation rate is approximately 0.12 grams of chemical oxygen demand (COD) per gram of volatile solids per day (i.e. 0.12 g COD/g VS/d). The efficiency of the hydrogen consuming methanogens can also be inferred by maintaining the hydrogen concentration in the biogas below 0.01% (10 ppm) and efficient removal of volatile fatty acids, specifically acetate and propionate.

The preferred biological parameters for anaerobic digestion are as follows:
(i) pH maintained between about 6.0 and 8.5, for example between 6.5 and 7.5;
(ii) Oxidation Reduction Potential (ORP) maintained at less than about −180 mV, for example −280 my;
(iii) Ammonia (Total Ammonium Nitrogen or “TAN”) maintained at less than about 3,000 mg/L, for example about 2,000 mg/L);
(iv) Conductivity maintained at less than about 27 mS/cm, for example about 22 mS/cm);
(v) Temperature maintained at about 55±2° C.;
(vi) Alkalinity maintained at less than about 15,000 mg calcium carbonate (CaCO₃)/L, for example about 12,000 mg CaCO₃/L); and
(vii) Total Dissolved Solids maintained at less than about 20,000 mg/L, for example about 15,000 mg/L.

The concentration of TAN is higher than that of many anaerobic digesters of the prior art and, in the process and method of the present invention, is important for the development of the buffer system contained within the process liquor. The presence of ammonia (TAN) increases the pH of the liquor and the solubility of carbon dioxide gas, which forms the basis of the carbonic acid—hydrogen carbonate buffer system. A high TAN concentration is required to establish the significant quantity of buffer necessary to provide stable operation during the period of acidification (10.5 g/L acetate; 15.0 g/L volatile fatty acids) that occurs during the initial days of the thermophilic high-solids batch anaerobic digestion. The high TAN concentration requires careful monitoring and control as free-ammonia is inhibitory to methanogens, particularly at elevated temperature. The high TAN concentration also results in the alkalinity of the process liquor being higher than that of many anaerobic digesters of the prior art.

In addition, the anaerobic culture is ‘starved’. For example, the culture may need to be set aside without introduction of food, between uses, to ensure volatile fatty acids (VFA) exhaustion, in particular the exhaustion of propionate, if this is not occurring in regular operation. Microbial propionate metabolism is thermodynamically unfavourable when acetate is present at any significant concentration. Consequently, propionate can only be anaerobically consumed, or depleted, under conditions of microbial starvation. During the early stages of a typical batch anaerobic digestion, acetate is present in relatively high concentrations (>10 mM; >600 mg/L) and, as a consequence, propionate degradation is inhibited resulting in the accumulation of propionate within the process water. The accumulated propionate can only be degraded toward the end of the batch digestion, once acetate has been significantly depleted. Before the process water can be reused in a subsequent batch, as a minimum, the propionate concentration must be reduced to the same concentration as it was at the start of the batch. Should propionate depletion between batches not be achieved, propionate will continue to accumulate, during subsequent batches, to concentrations that are inhibitory to methanogenesis, resulting in reactor acidification and ultimately process failure.

### Example 2

### Microbial Community Profiling by Terminal Restriction Fragment Length Polymorphism (T-RFLP)

T-RFLP is a molecular method that allows the microbial community to be explored relatively quickly and community profiles can be compared in samples collected at different time points. DNA is extracted from the sample, the 16S gene amplified selectively using a primer pair with a fluorescent label, the PCR product is digested with a restriction endonuclease which cuts at a specific sequence (4 bp enzymes are used as they cut most frequently, every 256 bp), the digested FOR product is run on a capillary sequencer which allows the terminal labelled fragments only to be sized precisely. The resulting profiles can give information about microbial identity (fragment size) and abundance (peak area).

The predominant methanogens in the anaerobic phase of the process of International Patent Application PCT/AU00/00865 (WO01/05729) have been cloned and sequenced and sequencing has identified several pure isolates.

### Methanogens

Sequencing has identified four methanogens from the anaerobic phase. The most predominant methanogens in the liquid phase were Methanoculleus species (chikugoensis and submarinus), with Methanothermobacter wolfeii present in lower numbers. In the solid phase, only one clone type was identified, the acetoclastic Methanosarcina thermophila. Another methanogen was isolated from the solid phase, purified and identified by sequencing as Methanoculleus thermophilus. Using the primers Arch f364FAM and Arch r1386 and the restriction enzyme Hae III (recognition site GGCC), the following size fragments would be expected: Methanoculleus spp. III; Methanothermobacter wolfeii 185; and Methanosarcina thermophila 115. Clones and pure cultures were used as T-RFLP templates and the size fragments obtained matched the predicted sizes. Preliminary T-RFLP profiles of archaea (methanogens) in four samples from the anaerobic digestion phase were obtained following Hae III digestion. The largest peak, or most predominant group of methanogens, in all four samples was attributed to Methanoculleus spp. Peaks attributed to Methanosarcina thermophila were also present in the four samples but were much smaller (approximately 10% the level of that attributed to Methanoculleus spp). They increased in size over time and by Day 10 had reached levels half the size of that of Methanoculleus spp. To give larger separation between these two groups, two different restriction enzymes were used (Taq I and Alu I) in a subsequent trial.

Samples were collected daily during an entire run of the process of International Patent Application PCT/AU00/00865 (WO 01/05729) (both aerobic and anaerobic phases) along with samples from the recycled anaerobic liquid and the solid portion of the reactor. DNA was extracted from the samples and population changes examined by T-RFLP. Fragment sizes below 50 bp are commonly excluded as they can arise from primers and primer dimers (Osborne, C. A., Rees, G. N., Bernstein, Y. and Janssen P. H. (2006). New Threshold and Confidence Estimates for Terminal Restriction Fragment Length Polymorphism Analysis of Complex Bacterial Communities. Applied and Environmental Microbiology, 72:1270-1278).

Consistently, the largest peak was at 89 bp, which was assigned to members of the Methanomicrobiaceae family (eg. Methanoculleus). This confirmed that Methanoculleus spp. predominate in the liquid of the anaerobic phase of the process of International Patent Application PCT/AU00/00865 (WO 01/05729) and also suggested that Methanoculleus survives in the aerobic phase. These species also predominated in the recycled anaerobic liquid, so are likely to be transferred in large amounts when the aerobic phase is flooded. A fragment of 148 bp, assigned to several methanogenic groups (Methanofollis/Methanocalculus/Methanospirillum) was present throughout the aerobic phase, increasing in proportion to higher levels than Methanoculleus spp. by Day 4. In the anaerobic phase, the 148 fragment disappeared quickly, to be replaced in Day 6 by another group of methanogens within the Methanomicrobiales genus, with a fragment size of 249. Another peak at 160 (Methanomicrobium/Methanogenium/Methanoplanus) appeared in large amounts in the anaerobic phase. On Day 7 this peak was approximately half the level of the Methanoculleus population and by Day 9 levels were higher than Methanoculleus. Methanosarcina species (367 bp) were only present at low levels in Day 0 and 1 of the aerobic phase and then appeared again at Day 10 (˜10% of the known methanogens), declining during the remainder of the anaerobic phase Day 11 (˜5%), and Day 12 (˜2%). In the only solid sample that was amplified (Day 9), Methanosarcina was the predominant methanogen, with Methanoculleus at only around 10%. This confirmed the results found by cloning and sequencing of the solid material. Methanothermobacter spp. (270) was found in high levels in one sample (Day 1) (˜60% of known methanogens). As it was not present in any of the other samples, this may have resulted from a clump of cells. Other fragment sizes, which could not be assigned to any known methanogens, were also found at low levels, which may represent unique methanogens.

The T-RFLP profiles of the methanogens using the second restriction enzyme, Alu I, confirmed the above findings. Methanoculleus spp. were the most predominant known methanogens and Methanosarcina thermophila the predominant methanogen in the solid material (42%). A few additional archaeal species could be identified with Alu I. This enzyme improved the detection of Methanothermobacter spp., as the cut site is highly conserved. Methanothermobacter levels were highest at the start of the aerobic phase (7%) and present at low levels (1%) thereafter. Two other large peaks were identified in the anaerobic phases but could not be assigned to any known methanogen.

It is envisaged that some new or fresh microorganisms may be introduced in addition to, or to supplement, the recycled or reused populations from prior anaerobic digestion steps. The OFMSW itself is one such source of methanogens. Consequently, within the reactor during the initial aerobic step, careful control of oxygen concentration and the resulting temperature to which the organic material will self-heat is required. Ideally, during the initial aeration period, the temperature of the organic material should be maintained above 50° C. but below 70° C., preferably below 65° C., and still preferably below 60° C.

As can be seen from the foregoing description, the method for the treatment of organic waste of the present invention, provides for the efficient operation of the anaerobic digestion step through management of the populations of microorganisms required in that step. This proactive management is only possible as a result of the decision to pursue identification of the important microorganisms and the phases, being liquid or solid, in which they are respectively generally present. The efficient operation of the method of the present invention is generally evident in relatively short digestion times and longevity of the process, in terms of how many times the cycle of aerobic and anaerobic steps may be repeated.

Modifications and variations such as would be apparent to the skilled addressee are considered to fall within the scope of the present invention.

1. A method for the treatment of organic waste, the method comprising alternating steps of anaerobic digestion and aerobic composting conducted in a single reactor vessel, wherein at or about the completion of the anaerobic digestion step at least a portion of any free draining fluid from the reactor vessel is directed for reuse in subsequent anaerobic digestion steps, and solids remaining in the reactor vessel from the anaerobic digestion step are subjected to a dewatering step from which a liquid is obtained that is ultimately also directed, at least in part, for reuse in subsequent anaerobic digestion steps.

2. A method according to claim 1, wherein both the free draining fluid from the reactor vessel and the liquid obtained from the dewatering step contain methanogenic microorganisms that contribute to the anaerobic digestion of organic waste.

3. A method according to claim 1 or 2, wherein the free draining fluid contains hydrogen consuming microorganisms.

4. A method according to any one of the preceding claims, wherein the liquid obtained from the dewatering step contains acetate consuming microorganisms.

5. A method according to any one of claims 2 to 4, wherein the methanogenic microorganisms contained in the free draining liquid includes at least one Methanoculleus species.

6. A method according to claim 5, wherein the at least one Methanoculleus species includes at least one of Methanoculleus thermophilus, Methanoculleus chikugoensis and Methanoculleus submarinus.

7. A method according to claim 5 or 6, wherein the free draining liquid further includes at least one Methanothermobacter or Methanobacterium species.

8. A method according to claim 7, wherein the free draining liquid includes the species Methanothermobacter wolfeii.

9. A method according to any one of claims 2 to 8, wherein the methanogenic microorganisms contained in the liquid obtained from the dewatering step include at least the species Methanosarcina thermophila.

10. A method according to claim 9, wherein the methanogenic microorganisms contained in the liquid obtained from the dewatering step further includes the species Methanoculleus thermophilus.

11. A method according to any one of the preceding claims, wherein the free draining fluid from the reactor vessel and the liquid obtained from the dewatering step are stored separately.

12. A method according to any one of the preceding claims, wherein the Total Ammonium Nitrogen concentration during anaerobic digestion is maintained at less than about 3,000 mg/L.

13. A method according to claim 12, wherein the Total Ammonium Nitrogen concentration during anaerobic digestion is maintained at about 2,000 mg/L.

14. A method for the management of biology in a batch process, wherein the batch process is an anaerobic digestion process and at or about the completion of a first anaerobic digestion step at least a portion of any free draining fluid from the reactor vessel in which the anaerobic digestion step is conducted is directed for reuse in subsequent anaerobic digestion steps, and solids remaining in the reactor vessel from the anaerobic digestion step are subjected to a dewatering step from which a liquid is obtained that is ultimately also directed, at least in part, for reuse in subsequent anaerobic digestion steps.

15. A method according to claim 14, wherein both the free draining fluid from the reactor vessel and the liquid obtained from the dewatering step contain methanogenic microorganisms that contribute to the anaerobic digestion of organic waste.

16. A method according to claim 14 or 15, wherein the free draining fluid contains hydrogen consuming microorganisms.

17. A method according to any one of claims 14 to 16, wherein the liquid obtained from the dewatering step contains acetate consuming microorganisms.

18. A method according to any one of claims 14 to 17, wherein the methanogenic microorganisms contained in the free draining liquid includes at least one Methanoculleus species.

19. A method according to claim 18, wherein the at least one Methanoculleus species includes at least one of Methanoculleus thermophilus, Methanoculleus chikugoensis and Methanoculleus submarinus.

20. A method according to claim 18 or 19, wherein the free draining liquid further includes at least one Methanothermobacter or Methanobacterium species.

21. A method according to claim 20, wherein the free draining liquid includes the species Methanothermobacter wolfeii.

22. A method according to any one of claims 14 to 21, wherein the methanogenic microorganisms contained in the liquid obtained from the dewatering step include at least the species Methanosarcina thermophila.

23. A method according to claim 22, wherein the methanogenic microorganisms contained in the liquid obtained from the dewatering step further includes the species Methanoculleus thermophilus.

24. A method according to any one of claims 14 to 23, wherein the Total Ammonium Nitrogen concentration during anaerobic digestion is maintained at less than about 3,000 mg/L.

25. A method according to claim 24, wherein the Total Ammonium Nitrogen concentration during anaerobic digestion is maintained at about 2,000 mg/L.

26. A method according to any one of claims 14 to 25, wherein the free draining fluid from the reactor vessel and the liquid obtained from the dewatering step are stored separately.

27. A method according to any one of the preceding claims, wherein a portion of the dewatered solids remaining in the reactor vessel from anaerobic digestion is directed for reuse in subsequent anaerobic digestion steps.

28. A method according to claim 27, wherein between about 5 to 20% by weight of the dewatered solids remaining in the reactor vessel from anaerobic digestion are directed for reuse.

29. A method according to claim 28, wherein about 10% by weight of the dewatered solids remaining in the reactor vessel from anaerobic digestion are directed for reuse.
